(19) United States
(12) Patent Application Publication
(10) Pub. No.: US 2007/0292358 A1
(43) Pub. Date: Dec. 20, 2007

(54) STORAGE-STABLE EMULSION SPRAY PRODUCT

(76) Inventors: Winfried EMMERLING, Tornesch (DE); Pamela KAFTAN, Quickborn (DE)

Correspondence Address: PAUL & PAUL, 2000 MARKET STREET, PHILADELPHIA, PA, 19103-3229, US

(21) Appl. No.: 11/762,899

(22) Filed: Jun. 14, 2007

Related U.S. Application Data
(63) Continuation of application No. PCT/EP05/13156, filed on Dec. 8, 2005.

(30) Foreign Application Priority Data
Dec. 16, 2004 (DE) 102004061228.5

(51) Int. Cl. A61K 8/28; B65D 88/54

(52) U.S. Cl. 424/47; 424/66; 222/320

(57) ABSTRACT

A perspiration-inhibiting and/or deodorizing cosmetic product that encompasses a water-in-oil emulsion having at least one antiperspirant and/or deodorant active substance, at least one propellant, and an aerosol dispensing apparatus, the parts of the valve of the dispensing apparatus that come into contact with the emulsion being made of nonmetallic materials.

### CROSS-REFERENCE TO RELATED APPLICATIONS

This application is a continuation under 35 U.S.C. § 365 and 35 U.S.C. § 120 of International Application No. PCT/EP2005/013156, filed Dec. 8, 2005. This application also claims priority under 35 U.S.C. § 119 of German Application No. DE 10 2004 061 228.5, filed Dec. 16, 2004. Each application is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to a perspiration-inhibiting and/or deodorizing agent in the form of a water-in-oil emulsion containing antiperspirant and/or deodorant active substances, which agent is packaged in a dispensing apparatus for spraying as an aerosol.

An aerosol is a dispersed system in which a solid or a liquid is present in very finely distributed form in a gas. As a rule, the aerosol itself is generated only upon application, with the aid of a suitable spraying system, by the spraying of solutions, emulsions, or suspensions; spray cans, for example, in which a liquefied compressed gas serves as a propellant gas, can be used for this purpose. Upon opening of the pressure valve, the propellant/preparation mixture escapes through a fine nozzle, and the propellant evaporates and leaves behind the finely distributed sprayed material as an aerosol. Aerosol antiperspirant sprays are becoming increasingly popular in the toiletries sector. Common antiperspirant spray compositions exist as anhydrous suspensions of the powdered perspiration-reducing active substance, usually an aluminum salt, in addition to the propellant gas, in a liquid carrier, usually a relatively volatile oil such as cyclomethicone. For better suspension of the powdered active substance, the liquid carrier often also contains a thickening agent, for example, bentone gel. The suspension must be shaken before spraying. A disadvantage of these suspension aerosols is the risk that the valve orifices or nozzle orifices may clog at higher utilization concentrations of the salt. Attempts have therefore been made to spray the antiperspirant salt in dissolved form. The presentation of aqueous antiperspirant salt solutions in propellant-containing metal cans caused considerable corrosion problems in terms of aerosol packaging, however, so that even with lacquered spray cans, corrosion phenomena inevitably occurred on the can. Common deodorant spray compositions are present as anhydrous ethanol solutions. A disadvantage is that the possibility for incorporation of hydrous or water-soluble deodorant active substances that are not also soluble in ethanol is nonexistent or very limited. Here again, the addition of even small quantities of water results in corrosion phenomena on standard valves and indeed on lacquered spray cans.

(2) Description of Related Art, Including Information Disclosed Under 37 C.F.R. §§ 1.97 and 1.98

Attempts have also been made to diminish these corrosion risks by using water-in-oil emulsions in which the antiperspirant and/or deodorant active substance is dissolved in the internal aqueous phase (WO 20004/030641 A1, WO 96/24326 A1). The hope here was that the external oil phase would prevent contact between the internal, corrosively active aqueous phase and the can and valve. Despite intense efforts, however, it has so far not been possible to develop entirely satisfactory products. It was possible to suppress corrosion only briefly, but this did not represent a solution for the consumer product sector, in which in some cases several years can pass between manufacture and utilization of the last residues of product. Customers must be assured in this context that the product and application system are not subject to any modification such as corrosion, and will still function perfectly even after that amount of time. Internal experiments have shown that even with formulations based on a water-in-oil emulsion containing antiperspirant and/or deodorant active substances, corrosion may be observed on the standard valves that are used, in particular, on the metal valve springs.

Antiperspirant spray compositions based on silicone oil-containing water-in-oil or water-in-silicone-oil emulsions are known. WO 96/24326 A1 describes a preparation having 10 to 50% of a water-in-oil emulsion that contains an aluminum salt, and 50 to 90% of a propellant gas, in which context the preparation can be packaged in an aluminum can. The problem of the corrosive effect of such compositions on the constituents of the package and of the dispensing apparatus is not addressed in this Application. WO 94/22420 A1 describes aerosols based on silicone-containing water-in-oil microemulsions that form clear gels on the skin upon evaporation of the propellant gas. This document also does not disclose the existing corrosion problem.

### BRIEF SUMMARY OF THE INVENTION

The object of the present invention was to develop an antiperspirant and/or deodorant product based on an aqueous emulsion having antiperspirant and/or deodorant active substances and having a dispensing apparatus, which product exhibits improved storage stability. A further object was to develop an antiperspirant and/or deodorant product based on an aqueous emulsion having antiperspirant and/or deodorant active substances and having a dispensing apparatus, which product exhibits decreased corrosion properties.

It has now been discovered, surprisingly, that the existing corrosion problems can be overcome. Selection of the materials used for the valve of the dispensing apparatus is critically important in this context. Valves suitable according to the present invention are characterized in that they contain no springs, or flexible elements having return characteristics, whose contact surface with the cosmetic composition is made of metallic materials, or in that they contain no springs at all.

A subject of the present invention is, therefore, a perspiration-inhibiting and/or deodorizing cosmetic product that encompasses a water-in-oil emulsion having at least one antiperspirant and/or deodorant active substance, at least one propellant, and an aerosol dispensing apparatus, the parts of the valve of the dispensing apparatus that come into contact with the emulsion being made of nonmetallic materials.

### BRIEF DESCRIPTION OF THE SEVERAL VIEWS OF THE DRAWING(S)

Not Applicable

### DETAILED DESCRIPTION OF THE INVENTION

In a first preferred embodiment of the invention, the valve comprises a valve cone coated with a lacquer or with a polymeric plastic A and just such a flexible element having return characteristics, which element is arranged in such a way that after actuation is completed, the valve is returned into the closure position (=rest position of the valve). In a further preferred embodiment of the invention, the valve comprises a flexible element having return characteristics and/or a valve cone made of at least one plastic B, preferably an elastomeric plastic. Preferred elastomeric plastics are selected from Buna, in particular, Buna N, Buna 421, Buna 1602, and Buna KA 6712, neoprene, butyl, and chlorobutyl. In a further preferred embodiment of the invention, the flexible element having return characteristics can be embodied as a spiral spring or helical compression spring. In a further preferred embodiment of the invention, the flexible element having return characteristics can be embodied integrally with the valve cone and can comprise flexible legs. In a particularly preferred embodiment of the invention, the valve cone and flexible element having return characteristics are embodied similarly, equivalently, or identically to what is depicted in WO 89/08062 A1, FIG. 1, and the explanations pertinent thereto. Particularly preferred in this context is the Ariane M valve model, obtainable from the Seaquist Perfect Company, in which valve the flexible element having recovery characteristics is embodied in the form of four elastic legs, integrally with the valve cone.

Also preferred according to the present invention is a valve construction according to U.S. Pat. No. 4,471,893 A1.

In a further preferred embodiment of the invention, the dispensing apparatus comprises a springless valve such as the one, for example, that is the subject of US 2003/0102328.

All the valves utilized according to the present invention comprise an internally lacquered valve plate, the lacquer coating and valve material being compatible with one another. If aluminum valves are used according to the present invention, their valve plates can then be coated internally with, for example, Microflex lacquer. If tinplate valves are used according to the present invention, their valve plates can then be internally coated with, for example, polyethylene terephthalate (PET). The containers used, which can be made, e.g., of tinplate or aluminum, aluminum containers being preferred according to the present invention, must likewise be internally lacquered or coated in view of the corrosiveness of the water-in-oil emulsions utilized according to the present invention. An internal protective lacquer preferred according to the present invention is an epoxy-phenol lacquer such as the one obtainable, inter alia, under the designation Hoba 7407 P.

The water-in-oil emulsion of the perspiration-inhibiting and/or deodorizing product according to the present invention encompasses an oil phase that makes up preferably 1-60 wt %, particularly preferably 10-50 wt %, and extraordinarily preferably 15-35 wt %, based in each case on the total weight of the emulsion. The emulsifiers, according to the present invention, count neither among the oil phase nor among the water phase. In a further preferred embodiment of the invention, the oil phase is made up, in a proportion of at least 90 wt %, of oil components that are liquid at 20° C. Preferred oil components are selected from:
volatile silicone oils, which can be cyclic such as, for example, octamethylcyclotetrasiloxane, decamethylcyclopentasiloxane, and dodecamethylcyclohexasiloxane, as well as mixtures thereof such as those contained, for example, in the commercial products DC 244, 245, 344, and 345 of Dow Corning, or linear, for example, hexamethyldisiloxane (L₂), octamethyltrisiloxane (L₃), decamethyltetrasiloxane (L₄), any two- or three-member mixtures of L₂, L₃ and/or L₄ such as those contained, for example, in the commercial products DC 2-1184, Dow Corning® 200 (0.65 cSt), and Dow Corning® 200 (1.5 cSt) of Dow Corning;
nonvolatile higher-molecular-weight dimethylpolysiloxanes, obtainable commercially, e.g., under the designation Dow Corning® 190, Dow Corning® 200 Fluid, having viscosities in the range 5-100 cSt, preferably 5-50 cSt, or even 5-10 cSt, and Baysilon® 350 M;
the esters of linear or branched, saturated or unsaturated fatty alcohols having 2-30 carbon atoms with linear or branched, saturated or unsaturated fatty acids having 2-30 carbon atoms, which can be hydroxylated. These include 2-ethylhexyl palmitate (e.g., Cegesoft® C 24), hexyldecyl stearate (Eutanol® G 16), hexyldecyl laurate, isodecyl neopentanoate, isononyl isononanoate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2 butyl octanoate, diisotridecyl acetate, n butyl stearate, n-hexyl laurate, n-decyl oleate, oleyl oleate, oleyl erucate, erucyl oleate, erucyl erucate, ethylene glycol dioleate and dipalmitate;
the benzoic acid esters of linear or branched C₈₋₂₂ alkanols, e.g., the commercial products Finsolv® TN (C₁₂-C₁₅ alkyl benzoate), Finsolv® SB (isostearyl benzoate), and Finsolv® EB (ethylhexyl benzoate);
the C₈-C₂₂ fatty alcohol esters of univalent or polyvalent C₂-C₇ hydroxycarboxylic acids, in particular, the esters of glycolic acid, lactic acid, malic acid, tartaric acid, citric acid, and salicylic acid. Esters of this kind based on linear C_{12/15} alkanols, e.g., C₁₂-C₁₅ alkyl lactate, and of C_{12/13} alkanols branched in the 2-position, e.g., di-C₁₂-C₁₃ alkyl malate, may be obtained under the trademark Cosmacol® from Nordmann, Rassmann GmbH & Co, Hamburg, in particular, the commercial products Cosmacol® EMI, Cosmacol® ESI, and Cosmacol® ETI;
the addition products of ethylene oxide and/or propylene oxide with univalent or polyvalent C₃₋₂₀ alkanols such as butanol, butanediol, myristyl alcohol, and stearyl alcohol, e.g., PPG-14 butyl ether (Ucon Fluid® AP), PPG-9 butyl ether (Breox® B25), PPG-10 butanediol (Macol® 57), PPG-3 myristyl ether (Witconol® APM), and PPG-15 stearyl ether (Arlamol® E);
liquid paraffin oils, isoparaffin oils, e.g., the commercial products of the Permethyl® series, in particular, isododecane, isohexadecane, and isoeicosane, and synthetic hydrocarbons such as polyisobutene or polydecenes, and alicyclic hydrocarbons, for example, the commercial product 1,3-di-(2-ethylhexyl)cyclohexane (Cetiol® S);
the branched saturated or unsaturated fatty alcohols having 6-30 carbon atoms. These alcohols are often also referred to as Guerbet alcohols, since they are obtainable according to the Guerbet reaction. Particularly preferred alcohol oils are, for example, hexyldecanol (Eutanol® G), octyldodecanol, and 2-ethylhexyl alcohol;
mixtures of Guerbet alcohols and Guerbet alcohol esters, e.g., the commercial product Cetiol® PGL (hexyldecanol and hexyldecyl laurate).
the symmetrical, asymmetrical, or cyclic esters of carbonic acid with fatty alcohols, for example, glycerol carbonate, dicaprylyl carbonate (Cetiol® CC), or the esters of DE-OS 197 56 454;
triglycerides of linear or branched, saturated or unsaturated, optionally hydroxylated C₈₋₃₀ fatty acids. The use of natural oils, e.g., soybean oil, cottonseed oil, sunflower oil, palm oil, palm kernel oil, linseed oil, almond oil, castor oil, corn oil, olive oil, rapeseed oil, sesame oil, thistle oil, wheat germ oil, peach-kernel oil, and the liquid components of coconut oil and the like, can be particularly suitable. Also suitable, however, are synthetic triglyceride oils, in particular, capric/caprylic triglycerides, e. g. the commercial products Myritol® 318, Myritol® 331 (Cognis), or Miglyol® 812 (Hüls) having unbranched fatty acid radicals, as well as glyceryl triisostearin and the commercial products Estol® GTEH 3609 (Uniqema) or Myritol® GTEH (Cognis) having branched fatty acid radicals;
dicarboxylic acid esters of linear or branched C₂-C₁₀ alkanols, in particular, diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate;
di-n-alkyl ethers having a total of 12 to 36, in particular, 12 to 24 C atoms, e.g., di-n-octyl ether (Cetiol® OE), di-n-n-hexyl-n-octyl ether, and n-octyl-n-decyl ether.

Particularly preferred oils are the volatile cyclic silicone oils decamethylcyclopentasiloxane and dodecamethylcyclohexasiloxane, the volatile linear silicone oils hexamethyldisiloxane (L₂), octamethyltrisiloxane (L₃), and decamethyltetrasiloxane (L₄), as well as any two- and three-member mixtures of L₂, L₃ and/or L₄, volatile and nonvolatile linear silicone oils from the Dow Corning 200 Fluid series having viscosities of 0.65, 1.0, 1.5, and 5 cSt, the ester oils 2-ethylhexyl palmitate (e.g., Cegesoft® C 24), hexyldecyl laurate, 2-ethylhexyl stearate, isopropyl myristate, isopropyl palmitate, and 2-ethylhexyl laurate, the benzoic acid esters of linear or branched C₈₋₂₂ alkanols, in particular, the commercial product Finsolv® TN (C₁₂-C₁₅ alkyl benzoate), C₁₂-C₁₅ alkyl lactate, di-C₁₂-C₁₃ alkyl malate, PPG-14 butyl ether (Ucon Fluid® AP), the commercial products of the Permethyl® series, in particular, isododecane, isohexadecane, and isoeicosane, as well as polyisobutene and polydecenes, and mixtures of the aforesaid components.

It may be preferred according to the present invention to utilize mixtures of the aforesaid oils. Particularly preferred in this context are mixtures of two types of oil component, e.g., volatile silicone oil and ester oil. Oil mixtures that contain at least one volatile cyclic and/or linear silicone oil are particularly preferred. Oil mixtures that contain predominantly, i.e., in a proportion of more than 50 wt %, a volatile cyclic and/or linear silicone oil are extraordinarily preferred. Also preferred are oil mixtures that contain 60-95 wt %, particularly preferably 70-90 wt %, of at least one volatile cyclic and/or linear silicone oil in combination with 5-40 wt %, particularly preferably 10-30 wt %, of at least one ester oil, in particular, one of the aforesaid ester oils.

In a further preferred embodiment of the invention, an at least 80-wt % portion of the oil components exhibits a refractive index n_{D} from 1.39-1.51. It is particularly preferred if 5-40-50 wt %, extraordinarily preferably 10-12-25-30 wt %, of the oil components exhibit a refractive index n_{D} from 1.43-1.51, preferably 1.44-1.49, particularly preferably 1.45-0.47-1.485, at 20° C. (measured at λ=589 nm).

Further cosmetic products preferred according to the present invention are characterized in that 5-50 wt %, preferably 10-40 wt %, particularly preferably 12-25 wt %, of the oil components that are liquid at room temperature are selected from isopropyl myristate, isopropyl palmitate, isohexadecane, isoeicosane, PPG-14 butyl ether, PPG-15 butyl ether, 2-hexyldecanol, isostearyl benzoate, dimethicone PEG/PPG-20/23 benzoate, PPG-53 butyl ether, isostearyl lactate, isostearyl palmitate, hexyldecyl laurate, mixtures of hexyldecanol and hexyldecyl laurate, isocetyl palmitate, 2-octyldodecanol, polydecenes, isocetyl stearate, 2-ethylhexyl stearate, hexyldecyl stearate, 16-methyl-1-heptadecanol, diethylhexylcyclohexane, 2-ethylhexyl laurate, benzyl laurate, C₁₂-C₁₅ alkyl benzoate, octyldodecyl benzoate, C₁₂-C₁₅ alkyl lactate, dimethicone PEG-8 benzoate, PPG-5-buteth-7, PPG-2-isodeceth-12, polyphenylmethylsiloxanes, in particular, phenyltrimethicone, PPG-2-ceteareth-9, isostearyl-isostearate, di-C₁₂-C₁₃ alkyl malate, isododecane, polyisobutene, and glycereth-7 benzoate, as well as mixtures of these components. Extraordinarily preferred oil components are selected from C₁₂-C₁₅ alkyl benzoate (e.g., the commercial product Finsolv TN), octyldodecyl benzoate, C₁₂-C₁₅ alkyl lactate, phenyltrimethicones, di-C₁₂-C₁₃ alkyl malate, and polyisobutene.

The water-in-oil emulsion of perspiration-inhibiting and/or deodorizing products that are preferred according to the present invention encompasses a water phase that constitutes preferably 40-99 wt %, particularly preferably 50-90 wt %, and extraordinarily preferably 60-85 wt %, based in each case on the total weight of the propellant-gas-free emulsion. The emulsifiers, according to the present invention, count neither among the oil phase nor among the water phase. According to the present invention, water as well as all water-soluble ingredients, with the exception of the emulsifiers, count among the water phase.

Water-soluble ingredients preferred according to the present invention are antiperspirant active substances. Water-in-oil emulsions preferred according to the present invention contain at least one water-soluble antiperspirant active substance. Antiperspirant active substances preferred according to the present invention are the water-soluble astringent inorganic and organic salts of aluminum, zirconium, and zinc, or any mixtures of said salts. Particularly preferred antiperspirant active substances are selected from the aluminum chlorohydrates, for example, aluminum sesquichlorohydrate, aluminum chlorhydrex propylene glycol (PG) or polyethylene glycol (PEG), aluminum sesquichlorhydrex PG or PEG, aluminum dichlorhydrex PG or PEG, aluminum hydroxide, further selected from the aluminum zirconium chlorohydrates, such as aluminum zirconium trichlorohydrate, aluminum zirconium tetrachlorohydrate, aluminum zirconium-pentachlorohydrate, aluminum zirconium octachlorohydrate, the aluminum zirconium chlorohydrate glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAI(SO₄)₂·12 H₂O, alum), aluminum undecylenoyl collagen amino acid, sodium aluminum lactate+aluminum sulfate, sodium aluminum chlorohydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxyallantoinate sodium aluminum chlorohydroxylactate, zinc chloride, zinc sulfocarbolate, zinc sulfate, and zirconium chlorohydrate. “Water solubility” is understood according to the present invention as a solubility of at least 5 wt % at 20° C., i.e., that quantities of at least 5 g of the antiperspirant active substance are soluble in 95 g of water at 20° C. In a preferred embodiment, the composition contains an astringent aluminum salt, in particular, aluminum chlorohydrate, which is marketed, for example, in powder form as Micro Dry® by Reheis, in the form of an aqueous solution as Locron® L by Clariant, as Chlorhydrol® and in activated form as Reach® 501 by Reheis. An aluminum sesquichlorohydrate is offered by Reheis under the designation Reach® 301. The use of aluminum zirconium tetrachlorohydrex glycine complexes, which are marketed, e.g., by Reheis under the designation Rezal® 36G, can also be particularly advantageous according to the present invention. The antiperspirant active substances can be used as aqueous solutions. Preferred cosmetic products according to the present invention are characterized in that the water-in-oil emulsions contain at least one antiperspirant salt in a total quantity from 15-55 wt %, by preference 25-50 wt %, and in particular, 30-40 wt %, based in each case on the weight of the active substance per unit weight of the entire propellant-gas-free water-in-oil emulsion.

Particularly preferred cosmetic products according to the present invention are characterized in that the water-in-oil emulsions contain at least one antiperspirant salt and 5-50 wt %, preferably 10-40 wt %, particularly preferably 12-25 wt % of the oil components that are liquid at room temperature, selected from isopropyl myristate, isopropyl palmitate, isohexadecane, isoeicosane, PPG-14 butyl ether, PPG-15 butyl ether, 2-hexyldecanol, isostearyl benzoate, dimethicone PEG/PPG-20/23 benzoate, PPG-53 butyl ether, isostearyl lactate, isostearyl palmitate, hexyldecyl laurate, mixtures of hexyldecanol and hexyldecyl laurate, isocetyl palmitate, 2-octyldodecanol, polydecenes, isocetyl stearate, 2-ethylhexyl stearate, hexyldecyl stearate, 16-methyl-1-heptadecanol, diethylhexylcyclohexane, 2-ethylhexyl laurate, benzyl laurate, C₁₂-C₁₅ alkyl benzoate, octyldodecyl benzoate, C₁₂-C₁₅ alkyl lactate, dimethicone PEG-8 benzoate, PPG-5-buteth-7, PPG-2-isodeceth-12, polyphenylmethylsiloxanes, in particular, phenyltrimethicone, PPG-2-ceteareth-9, isostearyl isostearate, di-C₁₂-C₁₃ alkyl malate, isododecane, polyisobutene, and glycereth-7 benzoate, as well as mixtures of said components, particularly preferably selected from C₁₂-C₁₅ alkyl benzoate, octyldodecyl benzoate, C₁₂-C₁₅ alkyl lactate, phenyltrimethicone, di-C₁₂-C₁₃ alkyl malate, and polyisobutene. The remainder of the oil components that are liquid at room temperature is selected from volatile cyclic silicone oils, in particular, cyclopentasiloxane, cyclohexasiloxane, hexamethyldisiloxane, octamethyltrisiloxane, and decamethyltetrasiloxane, as well as mixtures thereof. The result of the aforesaid combinations of antiperspirant salts and the selected oil components is, surprisingly, that the emulsions leave behind on the skin only slight or indeed no visible residues, and also do not stain clothing. In addition, the preferred oil component mixtures in combination with the nonmetallic valves result, surprisingly, in a particularly advantageous spray pattern. Without wishing to be confined to this theory, it is presumed that the preferred oil components positively influence the rheological properties of the water-in-oil emulsions, so that disruptive influences do not occur in contact with the nonmetallic valves and valve parts.

Deodorant active substances preferred according to the present invention are odor absorbers, ion exchangers having a deodorizing action, germ-inhibiting agents, prebiotically active components, and enzyme inhibitors or, particularly preferably, combinations of the aforesaid active substances. Silicates serve as odor absorbers that simultaneously can also advantageously assist the rheological properties of the water-in-oil emulsions of the products according to the present invention. Among the silicates that are particularly advantageous according to the present invention are chiefly sheet silicates, and among them in particular, montmorillonite, kaolinite, illite, beidellite, nontronite, saponite, hectorite, bentonite, smectite, and talc. Further advantageous odor absorbers are, for example, zeolites, zinc ricinoleate, cyclodextrins, certain metal oxides such as, for example, aluminum oxide, and chlorophyll. They are used preferably in a total quantity from 0.1-10 wt %, particularly preferably 0.5-7 wt %, and extraordinarily preferably 1-5 wt %, based in each case on the weight of the water-in-oil emulsion. Preferred according to the present invention as germ-inhibiting or antimicrobial active substances are, in particular, organohalogen compounds as well as organohalides, quaternary ammonium compounds, and a number of plant extracts and zinc compounds. These include, among others, triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphen bromide, ammonium phenol sulfonate, benzalkonium halides, benzalkonium cetyl phosphate, benzalkonium saccharinate, benzethonium chloride, cetylpyridinium chloride, laurylpyridinium chloride, lauryliso-quinolinium bromide, methylbenzedonium chloride. Also usable are phenol, aryl alcohols such as, in particular, phenoxyethanol, 2-methyl-4-phenylbutan-2-ol, and 2-methyl-5-phenylpentan-1-ol, disodiumdihydroxyethylsulfosuccinyl undecylenate, sodium bicarbonate, zinc lactate, sodium phenolsulfonate, and zinc phenolsulfonate, ketoglutaric acid, terpene alcohols such as, for example, farnesol, chlorophyllin-copper complexes, α-monoalkylglycerol ethers having a branched or linear, saturated or unsaturated, optionally hydroxylated C₆-C₂₂ alkyl radical, particularly preferably α-(2-ethylhexyl)glycerol ether, available commercially as Sensiva SC 50 (from Schülke & Mayr), carboxylic acid esters, in particular, carboxylic acid monoesters of mono-, di- and triglycerol (in particular, glycerol monolaurate, diglycerol monocaprinate, diglycerol monolaurate, triglycerol monolaurate, and triglycerol-monomyristate), lantibiotics, and plant extracts (e.g., green tea and constituents of linden blossom oil). Further preferred deodorant active substances are selected from so-called prebiotically active components, which are to be understood, according to the present invention, as those components that inhibit only or at least predominantly the odor-forming microbes of the skin microflora, but not the desirable microbes, i.e. the non-odor-forming ones. Explicitly included herein are the active substances that are disclosed in Applications DE 10333245 and DE 10 2004 011 968 as being prebiotically active; these include conifer extracts, in particular, from the Pinaceae group, and plant extracts from the group of the Sapindaceae, Araliaceae, Lamiaceae, and Saxifragaceae, in particular, extracts from Picea spp., Paullinia sp., Panax sp., Lamium album, or Ribes nigrum, as well as mixtures of said substances.

Further preferred deodorant substances are selected from the perfume oils having a germ-inhibiting effect and from the Deosaft perfume oils that are obtained from the Symrise Company, formerly Haarmann & Reimer. The enzyme inhibitors include substances that inhibit the enzymes responsible for the decomposition of perspiration, in particular, arylsulfatase, β-glucuronidase, aminoacylase, the ester-cleaving lipases, and the lipoxygenases, e.g., trialkylcitric acid esters, in particular, triethyl citrate, or zinc glycinate. The deodorant active substances can be used both individually and in mixtures. Particularly preferred are phenoxyethanol, α-(2-ethylhexyl)glycerol ether, diglycerol monocaprinate, 2-methyl-4-phenylbutan-2-ol, mixtures of phenoxyethanol and α-(2-ethylhexyl)glycerol ether, and mixtures of aryl alcohols, in particular, phenoxyethanol, with α-(2-ethylhexyl)glycerol ether and diglycerol monocaprinate. The total quantity of the deodorant active substances in the compositions used according to the present invention is preferably 0.1-10 wt %, particularly preferably 0.2-7 wt %, in particular, 0.3-5 wt %, and extraordinarily preferably 0.4-1.0 wt %, based in each case on the total weight of the water-in-oil emulsion.

The water-in-oil emulsion of the perspiration-inhibiting and/or deodorizing product according to the present invention further contains at least one water-in-oil emulsifier. The at least one water-in-oil emulsifier is contained preferably in a quantity from 0.5-5 wt %, particularly preferably 1.0-2.5 wt %, based in each case on the total weight of the emulsion. A group of water-in-oil emulsifiers that is particularly preferred according to the present invention is the poly-(C₂-C₃) alkylene glycol-modified silicones, whose earlier INCI name was Dimethicone Copolyol, having the present INCI names PEG-x Dimethicone (where x=2-20, preferably 3-17, particularly preferably 11-12), Bis-PEG-y Dimethicone (where y=3-25, preferably 4-20), PEG/PPG a/b Dimethicone (where a and b, mutually independently, denote numbers from 2-30, preferably 3-30, and particularly preferably 12-20, in particular, 14-18), Bis-PEG/PPG-c/d Dimethicone (where c and d, mutually independently, denote numbers from 10-25, preferably 14-20, and particularly preferably 14-16), and Bis-PEG/PPG-e/f PEG/PPG g/h Dimethicone (where e, f, g, and h, mutually independently, denote numbers from 10-20, preferably 14-18, and particularly preferably 16). Particularly preferred are PEG/PPG-18/18 Dimethicone, which is available commercially in a 1:9 mixture with cyclomethicone as DC 3225 C or DC 5225 C, PEG/PPG-4/12 Dimethicone, which is obtainable under the designation Abil B 8852, and Bis-PEG/PPG-14/14 Dimethicone, which is available commercially in a mixture with cyclomethicone as Abil EM 97 (Goldschmidt), Bis-PEG/PPG-20/20 Dimethicone, which is available under the designation Abil B 8832, PEG/PPG-5/3 Trisiloxane (Silsoft 305), and PEG/PPG-20/23 Dimethicone (Silsoft 430 and Silsoft 440). Additional water-in-oil emulsifiers preferred according to the present invention are poly-(C₂-C₃) alkylene glycol-modified silicones that are hydrophobically modified with C₄-C₁₈ alkyl groups, particularly preferably Cetyl PEG/PPG-10/1 Dimethicone (previously: Cetyl Dimethicone Copolyol, obtainable as Abil EM 90 or, in a mixture of polyglyceryl-4 isostearate, Cetyl PEG/PPG-10/1 Dimethicone, and hexyl laurate, under the commercial designation Abil WE 09), also Alkyl Methicone Copolyols and Alkyl Dimethicone Ethoxy Glucosides.

Additional water-on-oil emulsifiers suitable according to the present invention are selected from substances of the general formula A—O—(CHR¹—X—CHR²—O—)ₐ—A′, where A and A′ represent the same or different hydrophobic organic radicals, a represents a number from 1 to 100, by preference 2 to 60, in particular, 5 to 40, X represents a single bond or the group>CHOR³, R¹ and R² represent a hydrogen atom or a methyl group and are selected so that the radicals do not simultaneously represent methyl, and R³ represents a hydrogen atom or a branched or unbranched, saturated or unsaturated alkyl or acyl group having 1 to 20 carbon atoms.

It is particularly preferred if the water-in-oil emulsifier or emulsifiers are selected so that the radicals A and A′ are selected from the group of the branched and unbranched, saturated and unsaturated alkyl and acyl radicals and hydroxyacyl radicals having 10 to 30 carbon atoms, and furthermore from the group of the hydroxyacyl groups joined to one another via ester functions, according to the pattern: OOC—R″—CR′H—(OOC—R″—CR′H)_{b}—OOC—R″—CHR′, where R′ is selected from the group of the branched and unbranched alkyl groups having 1 to 20 carbon atoms and R″ is selected from the group of the branched and unbranched alkylene groups having 1 to 20 carbon atoms, and b can assume values from 0 to 200.

Additional preferred water-in-oil emulsifiers are selected from

(1) saturated alcohols having 8-24 C atoms, in particular, having 16-22 C atoms, e.g., cetyl alcohol, stearyl alcohol, arachidyl alcohol, or behenyl alcohol or mixtures of said alcohols such as those obtained upon industrial hydrogenation of vegetable and animal fatty acids;
(2) ethoxylated alcohols and carboxylic acids having 8-24 C atoms, in particular, having 16-22 C atoms, that have an HLB value from 1-8;
(3) propoxylated alcohols and carboxylic acids having 8-24 C atoms, in particular, having 16-22 C atoms;
(4) partial esters of a polyol having 3-6 C atoms and saturated and/or unsaturated, branched and/or unbranched fatty acids having 8-24, in particular, 12-18 C atoms. Such partial esters are, for example, the monoglycerides of palmitic acid, stearic acid, and oleic acid, the sorbitan mono- and/or diesters, in particular, those of myristic acid, palmitic acid, stearic acid, or of mixtures of said fatty acids. Also to be mentioned here are the monoesters of trimethylolpropane, erythritol, or pentaerythritol, and saturated fatty acids having 14-22 C atoms. The technical monoesters that are obtained by esterification of 1 mol polyol with 1 mol fatty acid, and represent a mixture of monoesters, diesters, triesters, and if applicable unesterified polyol, are also usable.
(5) Polyglycerol esters of saturated and/or unsaturated, branched and/or unbranched alkanecarboxylic acids with a chain length from 8-24, in particular, 12-18 C atoms, having up to 10 glycerol units, by preference up to 3 glycerol units, and a degree of esterification from 1-10, by preference 1-5;
(6) mono- and/or polyglycerol ethers of saturated and/or unsaturated, branched and/or unbranched alcohols with a chain length from 8-30, in particular, 12-18 C atoms, having up to 10 glycerol units, by preference up to 3 glycerol units, and a degree of etherification from 1-10, by preference 1-5;
(7) propylene glycol esters of saturated and/or unsaturated, branched and/or unbranched alkanecarboxylic acids with a chain length from 8-24, in particular, 12-18 C atoms;
(8) methylglucose esters of saturated and/or unsaturated, branched and/or unbranched alkanecarboxylic acids with a chain length from 8-24, in particular, 12-18 C atoms;
(9) polyglycerol methylglucose esters of saturated and/or unsaturated, branched and/or unbranched alkanecarboxylic acids with a chain length from 8-24, in particular, 12-18 C atoms.
   It can be advantageous according to the present invention to utilize products with a low degree of ethoxylation (3-5 EO) or propoxylation, for example, polyethoxylated hydrogenated or non-hydrogenated castor oil or ethoxylated cholesterol.

Particularly preferred water-in-oil emulsifiers are glyceryl lanolate, glyceryl monostearate, glyceryl distearate, glyceryl monoisostearate, glyceryl monomyristate, glyceryl monooleate, diglyceryl monostearate, glyceryl monolaurate, glyceryl monocaprinate, glyceryl monocaprylate, diglyceryl monoisostearate, diglyceryl diisostearate, propylene glycol monostearate, propylene glycol monolaurate, sorbitan monoisostearate, sorbitan monolaurate, sorbitan monocaprylate, sorbitan sesquistearate, sorbitan monoisooleate, sucrose distearate, cetyl alcohol, stearyl alcohol, arachidyl alcohol, behenyl alcohol, isobehenyl alcohol, 2-ethylhexylglycerol ether, selachyl alcohol, chimyl alcohol, polyethylene glycol-(2) stearyl ether (steareth-2), glyceryl sorbitan stearate, polyglyceryl-4 isostearate, polyglyceryl-2 sesquiisostearate, PEG-7 hydrogenated castor oil, isostearyl diglyceryl succinate, PEG-5 cholesteryl ether, PEG-30 dipolyhydroxystearate, decaglyceryl heptaoleate, polyglyceryl-3 diisostearate, PEG-8 distearate, diglycerol dipolyhydroxystearate, glycerol isostearate, sorbitan isostearate, polyglyceryl-3 methylglucose distearate, polyethoxylated hydrogenated or non-hydrogenated castor oil, ethoxylated cholesterol, PEG-2 stearate, PEG-45/dodecylglycol copolymer, PEG-22/dodecylglycol copolymer, and Methoxy PEG-22/Dodecyl Glycol Copolymer. It is very particularly preferred if combinations of the aforementioned water-in-oil emulsifiers, in particular, a combination of two emulsifiers, are used.

It can be advantageous according to the present invention also to use at least one o/w emulsifier in addition to the at least one water-in-oil emulsifier.

The water-in-oil emulsion used according to the present invention can additionally contain ethanol. Ethanol is preferred, for example, when the refreshing effect evoked by the high water content of the water-in-oil emulsions according to the present invention is to be further enhanced.

In a further preferred embodiment, the water-in-oil emulsions according to the present invention contain at least one water-soluble polyol selected from the water-soluble polyvalent C₂-C₉ alkanols having 2-6 hydroxyl groups and water-soluble polyethylene glycols having 3-20 ethylene oxide units, as well as mixtures thereof. These components are preferably selected from 1,2-propylene glycol, 2-methyl-1,3-propanediol, glycerol, butylene glycols such as 1,2-butylene glycol, 1,3-butylene glycol, and 1,4-butylene glycol, pentylene glycols, hexanediols such as 1,6-hexanediol, hexanetriols such as 1,2,6-hexanetriol, 1,8-octanediol, dipropylene glycol, tripropylene glycol, diglycerol, triglycerol, erythritol, sorbitol, and mixtures of the aforesaid substances. Suitable water-soluble polyethylene glycols are selected from PEG-3, PEG-4, PEG-6, PEG-7, PEG-8, PEG-9, PEG-10, PEG-12, PEG-14, PEG-16, PEG-18, and PEG-20 as well as mixtures thereof, PEG-3 to PEG-8 being preferred. Sugars and certain sugar derivatives, such as fructose, glucose, maltose, maltitol, mannitol, inositol, sucrose, trehalose, and xylose can also be suitable according to the present invention. 1,2-Propylene glycol and glycerol are particularly preferred. The water-in-oil emulsions according to the present invention contain the water-soluble polyvalent C₂-C₉ alkanol having 2-6 hydroxyl groups and/or the water-soluble polyethylene glycol having 3-20 ethylene oxide units preferably, in total, in quantities from 0.5-25 wt %, particularly preferably 1-20 wt %, and extraordinarily preferably 3-15 wt %, based in each case on the entire water-in-oil emulsion.

The water-in-oil emulsions utilized according to the present invention can furthermore preferably contain one or more preservatives. Preservatives preferred according to the present invention are formaldehyde releasers (such as, for example, 1,3-dimethylol-4,4-dimethylhydantoin, INCI name DMDM Hydantoin, available from the Lonza Company, for example, under the commercial designation Glydant), iodopropyl butylcarbamates such as 3-iodo-2-propinyl butylcarbamate (available, e.g., from Lonza under the commercial designations Glycacil-L, Glycacil-S and/or as Dekaben LMB from Jan Dekker), parabens (i.e., p-hydroxybenzoic acid alkyl esters such as methyl-, ethyl-, propyl- and/or butylparaben), phenoxyethanol, ethanol, benzoic acid, dibromodicyanobutane (2-bromo-2-bromomethylglutarodinitrile), 2-bromo-2-nitropropane-1,3-diol, imidazolidinyl urea, 5-chloro-2-methyl-4-isothiazolin-3-one, 2-chloracetamide, benzalkonium chloride, benzyl alcohol, salicylic acid, and salicylates. Preservatives that are particularly preferred according to the present invention are selected from iodopropyl butylcarbamates, parabens (methyl-, ethyl-, propyl- and/or butylparaben), and/or phenoxyethanol. The preservatives are contained preferably in quantities from 0.01-2, particularly preferably 0.1-0.5, and extraordinarily preferably 0.2-1.0 wt %, based in each case on the weight of the water-in-oil emulsion.

The water-in-oil emulsions utilized according to the present invention can furthermore preferably contain a fragrance component. Individual odorant compounds, e.g., synthetic products of the ester, ether, aldehyde, ketone, alcohol, and hydrocarbon types, can be used as fragrances or perfume oils. Odorant compounds of the ester type are, for example, phenoxyethyl isobutyrate, benzyl acetate, p-tert.-butylcyclohexyl acetate, dimethylbenzylcarbinyl acetate, linalyl acetate, phenylethyl acetate, linalyl benzoate, ethyl-methylphenyl glycinate, benzyl formate, allylcyclohexyl propionate, styrallyl propionate, and benzyl salicylate. The ethers include, for example, benzylethyl ether; the aldehydes, for example, the linear alkanals having 8 to 18 C atoms, citral, citronellal, citronellyloxyacetaldehyde, cyclamenaldehyde, hydroxycitronellal, lilial, and bourgeonal; the ketones, for example, the ionones α-isomethylionone and methylcedrylketone; the alcohols, anethol, citronellol, eugenol, geraniol, linalool, phenylethyl alcohol, and terpineol; the hydrocarbons include principally the terpenes and balsams. Preferably, however, mixtures of different odorants are used which together produce an appealing fragrance note. Suitable perfume oils can also contain natural odorant mixtures such as those accessible from vegetable or animal sources, e.g., pine, citrus, jasmine, lily, rose, or ylang-ylang oil. Less-volatile essential oils that are usually utilized as flavor components are also suitable as perfume oils, e.g., sage oil, chamomile oil, clove oil, lemon balm oil, mint oil, cinnamon leaf oil, linden blossom oil, juniper berry oil, vetiver oil, olibanum oil, galbanum oil, and laudanum oil.

The fragrance component(s) are contained preferably in quantities from 0.01 to 4 wt %, particularly preferably 0.5-2 wt %, based in each case on the weight of the water-in-oil emulsion.

The water-in-oil emulsions used according to the present invention advantageously can further contain at least one skin-cooling active substance. Skin-cooling active substances that are suitable according to the present invention are, for example, menthol, isopulegol, and menthol derivatives, e.g., menthyl lactate, menthyl glycolate, menthylpyrrolidone carboxylic acid, menthylmethyl ether, menthoxypropanediol, menthone glycerin acetal (9-methyl-6-(1-methylethyl)-1,4-dioxaspiro(4.5)decane-2-methanol), monomenthyl succinate, and 2-hydroxymethyl-3,5,5-trimethylcyclohexanol. Preferred as skin-cooling active substances are menthol, isopulegol, menthyl lactate, menthoxypropanediol, and menthylpyrrolidone carboxylic acid, as well as mixtures of these substances, in particular, mixtures of menthol and menthyl lactate, menthol, menthol glycolate, and menthyl lactate, menthol and menthoxypropanediol, or menthol and isopulegol. The water-in-oil emulsions utilized according to the present invention contain at least one skin-cooling active substances preferably in quantities from 0.01-1 wt %, particularly preferably 0.02-0.5 wt %, and extraordinarily preferably 0.05-0.2 wt %, based in each case on the total weight of the water-in-oil emulsion. The water-in-oil emulsions utilized according to the present invention can furthermore preferably contain at least one plant extract. Plant extracts are usually produced by extraction of the entire plants but also, in individual cases, exclusively from blossoms and/or leaves and/or seeds and/or other plant parts. Especially preferred according to the present invention are the extracts from aloe vera, green tea, hamamelis, bamboo, chamomile, marigolds, heartsease, peony, horse chestnut, sage, willow bark, cinnamon tree, chrysanthemums, oak bark, nettles, hops, burdock root, horsetail, hawthorn, linden blossoms, almonds, fir needles, sandalwood, juniper, coconut, kiwi, guava, lime, mango, apricots, wheat, melon, orange, grapefruit, avocado, rosemary, birch, beech buds, malva, lady's-smock, yarrow, wild thyme, thyme, lemon balm, restharrow, hibiscus (Althaea), malva (Malva sylvestris), violets, blackcurrant leaves, coltsfoot, cinquefoil, ginseng, ginger root, and sweet potato. Algae extracts can also be used advantageously. The algae extracts utilized according to the present invention derive from green algae, brown algae, red algae, or blue-green algae (Cyanobacteria). The algae used for extraction can be both of natural origin and obtained by means of biotechnological processes, and if desired can be modified with respect to their natural form. The modification of the organisms can be performed by genetic engineering, by culturing, or by cultivation in media enriched with selected nutrients. Preferred algae extracts derive from kelp, blue-green algae, from the green alga Codium tomentosum, and from the brown alga Fucus vesiculosus. A particularly preferred algae extract derives from blue-green algae of the species Spirulina that have been cultivated in a magnesium-enriched medium. The water-in-oil emulsions utilized according to the present invention can also contain mixtures of multiple, in particular, two, different plant extracts. The water-in-oil emulsions utilized according to the present invention contain at least one plant extract, preferably in quantities from 0.01-5 wt %, particularly preferably 0.1-2 wt %, and extraordinarily preferably 0.5-1.0 wt %, based in each case on the total weight of the water-in-oil emulsion.

In a further preferred embodiment, the water-in-oil emulsions utilized according to the present invention contain at least one vitamin, provitamin, or compound referred to as a vitamin precursor, from the vitamin groups A, B, C, E, H, and K and the esters of the aforesaid substances. The group of substances referred to as vitamin A includes retinol (vitamin A₁) as well as 3,4-didehydroretinol (vitamin A₂). β-Carotene is the provitamin of retinol. Vitamin A components that are suitable according to the present invention are, for example, vitamin A acid and its esters, vitamin A aldehyde, and vitamin A alcohol, as well as its esters such as retinyl palmitate and retinyl acetate. The water-in-oil emulsions utilized according to the present invention contain the vitamin A component preferably in quantities from 0.05-1 wt % based on the entire water-in-oil emulsion. Members of the vitamin B group or vitamin B complex are, among others:
Vitamin B₁, trivial name: thiamine, chemical description: 3-[(4′-amino-2′-methyl-5′-pyrimidinyl)methyl]-5-(2-hydroxyethyl)-4-methylthiazolium chloride. Thiamine hydrochloride is preferably used in quantities from 0.05 to 1 wt %, based on the entire water-in-oil emulsion.
Vitamin B₂, trivial name: riboflavin, chemical description: 7,8-dimethyl-10-(1-D-ribityl)benzo[g]pteridine-2,4(3H,10H)-dione. Riboflavin or its derivatives are preferably used in quantities from 0.05 to 1 wt %, based on the entire water-in-oil emulsion.
Vitamin B₃. The compounds nicotinic acid and nicotinic acid amide (niacinamide) are listed under this designation. Nicotinic acid amide is preferred according to the present invention; it is contained in the agents according to the present invention preferably in quantities from 0.05 to 1 wt % based on the entire water-in-oil emulsion.
Vitamin B₅ (pantothenic acid and panthenol). Panthenol is preferably used. Derivatives of panthenol usable according to the present invention are, in particular, the esters and ethers of panthenol as well as cationically derivatized panthenols. In a further preferred embodiment of the invention, instead of and in addition to pantothenic acid or panthenol it is also possible to use derivatives of 2-furanone having the general structural formula (I)

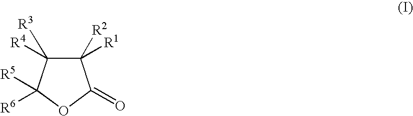

The 2-furanone derivatives in which the substituents R¹ to R⁶, mutually independently, represent a hydrogen atom, a hydroxyl radical, a methyl, methoxy, aminomethyl, or hydroxymethyl radical, a saturated or mono- or diunsaturated, linear or branched C₂-C₄ hydrocarbon radical, a saturated or mono- or diunsaturated, branched or linear mono-, di-, or trihydroxy-C₂-C₄ hydrocarbon radical, or a saturated or mono- or diunsaturated, branched or linear mono-, di-, or triamino-C₂-C₄ hydrocarbon radical, are particularly preferred. Particularly preferred derivatives are the substances (also available commercially) dihydro-3-hydroxy-4,4-dimethyl-2(3H)furanone having the trivial name pantolactone (Merck), 4-hydroxymethyl-γ-butyrolactone (Merck), 3,3-dimethyl-2-hydroxy-γ-butyrolactone (Aldrich), and 2,5-dihydro-5-methoxy-2-furanone (Merck), all stereoisomers expressly being included. The 2-furanone derivative that is extraordinarily preferred according to the present invention is pantolactone (dihydro-3-hydroxy-4,4-dimethyl-2(3H)-furanone), such that in formula (I) R¹ denotes a hydroxyl group, R² a hydrogen atom, R³ and R⁴ a methyl group, and R⁵ and R⁶ a hydrogen atom. The stereoisomer (R)-pantolactone occurs upon the breakdown of pantothenic acid.

The aforesaid compounds of the vitamin B₅ type, as well as the 2-furanone derivatives, are contained in the agents according to the present invention preferably in a total quantity from 0.05 to 5 wt %, particularly preferably 0.1 to 3 wt %, extraordinarily preferably 0.5 to 2 wt %, based in each case on the entire water-in-oil-emulsion.
Vitamin B₆; this is to be understood not as a uniform substance but as the derivatives, known by the trivial names pyridoxine, pyridoxamine, and pyridoxal, of 5-hydroxymethyl-2-methylpyridin-3-ol. Vitamin B₆ is contained in the agents according to the present invention preferably in quantities from 0.0001 to 1.0 wt %, in particular, 0.001 to 0.01 wt %, based in each case on the entire water-in-oil emulsion.
Vitamin B₇ (biotin), also known as vitamin H or “skin vitamin.” Biotin is (3aS,4S,6aR)-2-oxohexahydrothienol[3,4-d]imidazol-4-valeric acid. Biotin is contained in the agents according to the present invention preferably in quantities from 0.0001 to 1.0 wt %, in particular, 0.001 to 0.01 wt %, based in each case on the entire water-in-oil emulsion.
Vitamin C (ascorbic acid) Vitamin C is used preferably in quantities from 0.1 to 3 wt % based on the entire water-in-oil emulsion. Utilization of the derivatives ascorbyl palmitate, stearate, dipalmitate, and acetate, magnesium ascorbyl phosphate, sodium ascorbyl phosphate, sodium and magnesium ascorbate, disodium ascorbyl phosphate and sulfate, potassium ascorbyl tocopheryl phosphate, chitosan ascorbate, or ascrobyl glucoside can be preferred. Utilization in combination with tocopherols can likewise be preferred.
The vitamin E group includes tocopherols, in particular, α-tocopherol, and its derivatives. Preferred derivatives are, in particular, the esters such as tocopheryl acetate, nicotinate, phosphate, succinate, linoleate, oleate, tocophereth-5, tocophereth-10, tocophereth-12, tocophereth-18, tocophereth-50, and tocophersolan. Tocopherol and its derivatives are contained preferably in quantities from 0.05-1 wt %, based on the entire water-in-oil emulsion.
“Vitamin F” is usually understood to mean essential fatty acids, in particular, linoleic acid, linolenic acid, and arachidonic acid.
“Vitamin H” is another designation for biotin or vitamin B₇ (see above).
The fat-soluble vitamins of the vitamin K group, which are based on the fundamental structure of 2-methyl-1,4-naphthoquinone, include phylloquinone (vitamin K₁), farnoquinone or menaquinone-7 (vitamin K₂), and menadione (vitamin K₃). Vitamin K is contained preferably in quantities from 0.0001 to 1.0 wt %, in particular, 0.01 to 0.5 wt %, based in each case on the entire water-in-oil emulsion.

Vitamin A palmitate (retinyl palmitate), panthenol, pantolactone, nicotinic acid amide, pyridoxine, pyridoxamine, pyridoxal, biotin, ascorbyl palmitate and acetate, Mg ascorbyl phosphate, sodium ascorbyl phosphate, sodium and magnesium ascorbate, and the tocopherol esters, in particular, tocopheryl acetate, are particularly preferred according to the present invention.

In a further preferred embodiment, the water-in-oil emulsions utilized according to the present invention contain at least one skin-soothing active substance. Skin-soothing substances that are preferred according to the present invention are selected from allantoin, α-bisabolol, α-lipoic acid, and (2-hydroxyethyl)urea.

Particularly preferred cosmetic products according to the present invention are characterized in that they contain at least one skin-soothing active substance in a total quantity from 0.001 to 5 wt %, particularly preferably 0.01 to 2 wt %, and extraordinarily preferably 0.1 to 1 wt %, based in each case on the entire water-in-oil emulsion.

Cosmetic compositions based on water-in-oil emulsions that contain antiperspirant salts can act to dry out the skin. It has been found, surprisingly, that by means of the addition of selected active substances, an unexpected compensation for the negative influence on skin moisture content, and in fact a skin-moisturizing action, can be achieved for the water-on-oil emulsions utilized according to the present invention. Further cosmetic products preferred according to the present invention are therefore characterized in that the water-in-oil emulsion contains at least one moisture-donating active substance, selected from panthenol, pantolactone, deoxy sugars, particularly preferably rhamnose and fucose, polysaccharides that contain at least one deoxy sugar module, urea, N,N′-bis(2-hydroxyethyl)urea, betaine (Me₃N⁺—CH₂—COO⁻), glycosaminoglycans, particularly preferably hyaluronic acid, dextran, dextran sulfate, chondroitin 4-sulfate, and chondroitin 6-sulfate, as well as any mixtures of these substances. Particularly preferred are products according to the present invention that contain in the water-in-oil emulsion at least one antiperspirant salt and at least one moisture-donating active substance selected from panthenol, pantolactone, deoxy sugars, particularly preferably rhamnose and fucose, polysaccharides that contain at least one deoxy sugar module, urea, N,N′-bis(2-hydroxyethyl)urea, betaine (Me₃N⁺—CH₂—COO⁻), glycosaminoglycans, particularly preferably hyaluronic acid, dextran, dextran sulfate, chondroitin 4-sulfate, and chondroitin 6-sulfate, as well as any mixtures of said substances.

Particularly preferred cosmetic products according to the present invention are characterized in that they contain at least one skin-moisturizing active substance selected from panthenol, pantolactone, deoxy sugars, particularly preferably rhamnose and fucose, polysaccharides that contain at least one deoxy sugar module, urea, N,N′-bis(2-hydroxyethyl)urea, betaine (Me₃N⁺—CH₂—COO⁻), glycosaminoglycans, particularly preferably hyaluronic acid, dextran, dextran sulfate, chondroitin 4-sulfate, and chondroitin 6-sulfate, as well as any mixtures of said substances, in a total quantity from 0.001 to 5 wt %, particularly preferably 0.01 to 2 wt %, and extraordinarily preferably 0.1 to 1 wt %, based in each case on the entire water-in-oil emulsion. Products according to the present invention having a combination of at least one antiperspirant salt based on aluminum and/or aluminum/zirconium compounds and an active substance selected from panthenol, pantolactone, and N,N′-bis(2-hydroxyethyl)urea, in particular, the combinations aluminum chlorohydrate and panthenol, aluminum chlorohydrate and pantolactone, aluminum chlorohydrate and N,N′-bis(2-hydroxyethyl)urea, aluminum zirconium tetrachlorohydrex glycine complex and panthenol, aluminum zirconium tetrachlorohydrex glycine complex and pantolactone, zirconium tetrachlorohydrex glycine complex and N,N′-bis(2-hydroxyethyl)urea, are extraordinarily preferred.

Further particularly preferred products according to the present invention are characterized in that the water-in-oil emulsion contains at least one dimethiconol (S1). It has been found, surprisingly, that the addition of a dimethiconol improves the spray pattern of the emulsion. This is preferably to be understood to mean that the spray does not become too greatly atomized, i.e. that the spray droplets are not too small and do not remain in the air without arriving at the skin surface that is to be treated. Also to be understood as an improvement of the spray pattern is the fact that the emulsion remains on the skin after being sprayed on, and does not “bounce off.” It has also been found, surprisingly, that the addition of a dimethiconol greatly reduces, even in a context of longer periods of time between two spray actuations, a clogging or sticking of the spray device that can possibly occur. The dimethiconols preferred for the water-in-oil emulsions of the products according to the present invention can be linear, branched, cyclic, or cyclic with branches. Preferred linear dimethiconols can be represented by the following structural formula (S1-I):
(SiOHR¹₂)—O—(SiR²₂—O—)ₓ—(SiOHR¹₂) (S1-I)

Preferred branched dimethiconols can be represented by the structural formula (S1-II): 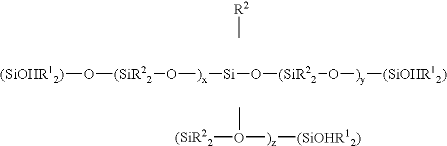
The radicals R¹ and R² each denote, mutually independently, hydrogen, a methyl radical, a C₂-C₃₀ linear, saturated or unsaturated hydrocarbon radical, a phenyl radical, and/or an aryl radical. Non-limiting examples of the radicals represented by R¹ and R² include alkyl radicals such as methyl, ethyl, propyl, isopropyl, butyl, isobutyl, pentyl, isopentyl, neopentyl, amyl, isoamyl, hexyl, isohexyl, and the like; alkenyl radicals such as vinyl, halovinyl, alkyl vinyl, allyl, haloallyl, alkyl allyl; cycloalkyl radicals such as cyclobutyl, cyclopentyl, cyclohexyl, and the like; phenyl radicals, benzyl radicals, halogenated hydrocarbon radicals such as 3-chloropropyl, 4-bromobutyl, 3,3,3-trifluoropropyl, chlorocyclohexyl, bromophenyl, chlorophenyl, and the like, as well as sulfur-containing radicals such as mercaptoethyl, mercaptopropyl, mercaptohexyl, mercaptophenyl, and the like; by preference, R¹ and R² are an alkyl radical that contains 1 to approximately 6 carbon atoms, and most preferably R¹ and R² are methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. Methyl, phenyl, and C₂ to C₂₂ alkyl radicals are preferred as R¹ and R². Lauryl, stearyl, and behenyl are very particularly preferred for the C₂-C₂₂ alkyl radicals. The numbers x, y, and z are whole numbers and each extend, mutually independently, from 0 to 50,000. The molar weights of the dimethiconols are between 1,000 D and 10,000,000 D. The viscosities are between 100 and 10,000,000 cPs, measured at 25° C. using a glass capillary viscosimeter according to Dow Corning Corporate Test Method CTM 0004 of Jul. 20, 1970. Preferred viscosities are between 1,000 and 5,000,000 cPs, very particularly preferred viscosities are between 10,000 and 3,000,000 cPs. The most preferred range is between 50,000 and 2,000,000 cPs.
Dimethiconols particularly preferred for use are added to the water-in-oil emulsions preferred according to the present invention not in pure form, but in dissolved form, for example, as a solution in a cyclomethicone or a dimethicone. The teaching of the present invention of course also encompasses the fact that the dimethiconols can be added to the water-in-oil emulsions preferred according to the present invention in already-preemulsified form. The corresponding (pre)emulsion of the dimethiconols can be produced both from the corresponding dimethiconols after manufacture thereof and in accordance with the usual methods for emulsification known to one skilled in the art. Anionic, nonionic, or zwitterionic surfactants, and emulsifiers as auxiliaries, can be used as adjuvants for manufacture of the corresponding emulsions. The emulsions of the dimethiconols can, of course, also be manufactured directly by way of an emulsion polymerization reaction. Such methods are also well known to one skilled in the art. Reference may be made here, for example, to the “Encyclopedia of Polymer Science and Engineering,” Volume 15, Second Edition, pages 204 to 308, John Wiley & Sons, Inc. 1989. Reference is expressly made to this standard work. When the dimethiconols are used as an emulsion, the droplet size of the emulsified particles is then, according to the present invention, 0.01 μm to 10,000 μm, preferably 0.01 to 100 μm, very particularly preferably 0.01 to 20 μm, and most preferably 0.01 to 10 μm. The particle size is determined, in this context, using the light diffusion method. When branched dimethiconols are used, this is understood to mean that the branching is greater than a random branching that occurs randomly as a result of contaminants of the respective monomers. For purposes of the present compound, “branched dimethiconols” is therefore to be understood to mean that the degree of branching is greater than 0.01%. A degree of branching greater than 0.1% is preferred, and one greater than 0.5% is very particularly preferred. The degree of branching is determined, in this context, from the ratio of the unbranched monomers (i.e., the quantity of monofunctional siloxane) to the branching monomers (i.e., the quantity of tri- and tetrafunctional siloxanes). Both less-branched and highly-branched dimethiconols can be very particularly preferred according to the present invention. The following commercial products are recited as examples of products of this kind: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all the aforesaid Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all the aforesaid Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all the aforesaid Crompton Corporation), SM555, SM2725, SM2765, SM2785 (all the aforesaid GE Silicones), Taylor T-Sil CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Particularly preferred according to the present invention, therefore, is a perspiration-inhibiting and/or deodorizing cosmetic product that encompasses a water-in-oil emulsion having at least one antiperspirant and/or deodorant active substance and at least one dimethiconol, at least one propellant, and an aerosol dispensing apparatus, the parts of the valve of the dispensing apparatus that come into contact with the emulsion being made of nonmetallic materials.

Further products particularly preferred according to the present invention are characterized in that they contain at least one dimethiconol (S1) in a total quantity from 0.001 to 5 wt %, by preference 0.01 to 2 wt %, particularly preferably 0.1 to 1.0 wt %, and in particular, 0.2 to 0.5 wt %, based on the weight of the active substance per unit weight of the (propellant-gas-free) water-in-oil emulsion.

Propellants (propellant gases) that are suitable according to the present invention are propane, propene, n-butane, isobutane, isobutene, n-pentane, pentene, isopentane, isopentene, methane, ethane, dimethyl ether, nitrogen, air, oxygen, nitrous oxide, 1,1,1,2-tetrafluoroethane, heptafluoro-n-propane, perfluorethane, monochlorodifluoromethane, 1,1-difluoroethane, both individually and in combination. Hydrophilic propellant gases such as, for example, carbon dioxide can also be used advantageously for purposes of the present invention if the selected proportion of hydrophilic gases is low, and lipophilic propellant gas (e.g., propane/butane) is present in excess. Propane, n-butane, isobutane, and mixtures of these propellant gases are particularly preferred. It has been shown that the use of n-butane as a sole propellant gas can be particularly preferred according to the present invention. The quantity of propellant is preferably 10-90 wt %, particularly preferably 40-90 wt %, and extraordinarily preferably 50-80 wt %, based in each case on the total weight of the preparation comprising the water-in-oil emulsion and the propellant.

Suitable compressed-gas containers are vessels made of metal (aluminum, tinplate, tin), protected or non-shattering plastic, or glass that is coated externally with plastic, in the selection of which pressure resistance and resistance to breakage, corrosion resistance, and ease of filling, as well as aesthetic considerations, handling, imprintability, etc. play a role. Special internal protection lacquers ensure corrosion resistance with respect to the water-in-oil emulsions.

Because of the specific selection of the valve parts, the perspiration-inhibiting products according to the present invention exhibit particularly high corrosion resistance despite the water phase in the aerosol container, which represents a great advantage over the existing art. The water-in-oil emulsions used according to the present invention furthermore exhibit outstanding skin compatibility. A particular advantage is that the sprayed products are notable, on the skin, for a pleasant, non-sticky skin feel. The water content produces a definite fresh feeling after application.

The examples below are intended to explain the subject matter of the invention without limiting it thereto.

| | Example 1 | | Example 2 | |
|---|---|---|---|---|
| | [wt % based on the | Example 1 | [wt % based on the | Example 2 |
| | entire preparation | [wt % based on | entire preparation | [wt % based on |
| | including | water-in-oil | including | water-in-oil |
| Constituent | propellant gas] | emulsion] | propellant gas] | emulsion] |
| Dow Corning | 2.0 | 12.5 | 1.0 | 4.9 |
| 345 Fluid | | | | |
| 1,2-Propylene | 0.5 | 3.1 | 2.0 | 9.8 |
| glycol | | | | |
| Dow Corning | 3.0 | 16.9 (oil) | 2.5 | 11.0 (oil) |
| 5225 C | | 1.9 (emulsifier) | | 1.2 (emulsifier) |
| 2-Ethylhexyl | 0.5 | 3.1 | 0.5 | 2.4 |
| palmitate | | | | |
| Phenoxyethanol | 0.08 | 0.5 | 0.1 | 0.5 |
| Water, deionized | 4.92 | 30.7 | 7.15 | 34.9 |
| Microdry | 5.0 | 31.3 | 7.25 | 35.3 |
| n-Butane | 84.0 | — | 79.5 | — |

Composition Examples 1 and 2 were introduced into an aluminum can that was coated internally with an epoxy-phenol lacquer and was equipped with the Ariane M valve, obtainable from the Seaquist Perfect Company, and a valve plate coated internally with Microflex lacquer, and stored for 12 weeks at 45° C. As compared with the same product that was equipped with a valve not according to the present invention, which had a metallic contact surface with the water-in-oil emulsion, the product according to the present invention exhibited no signs of corrosion on the valve parts at the end of the storage test. The product not according to the present invention had definite signs of corrosion on the valve spring at the end of the 12-week storage test at 45° C.

Further Examples of Formulations.

| | Example No. | | | | | | | | | |
|---|---|---|---|---|---|---|---|---|---|---|
| | 3 | 4 | 5 | 6 | 7 | 8 | 9 | 10 | 11 | 12 |
| Dow Corning 345 Fluid | 16.7 | 12.9 | 17.6 | 10.2 | 12.2 | 16.4 | 21.2 | 9.0 | 18.8 | 13.1 |
| Dow Corning 200 Fluid, 5 cst | / | 10.4 | / | 8.5 | 9.0 | / | / | 7.5 | / | 6.4 |
| 1,2-Propylene glycol | 9.1 | / | 2.4 | / | 8.6 | / | 8.6 | / | 6.0 | 7.2 |
| Dipropylene glycol | / | 7.4 | / | 9.6 | / | 2.4 | / | 17.0 | / | / |
| PEG/PPG-18/18 Dimethicone | 1.4 | 1.4 | 1.4 | 1.1 | 1.4 | 1.2 | 1.4 | 1.0 | 1.6 | 1.5 |
| Ethylhexyl palmitate | 2.3 | / | 2.4 | 1.5 | 2.3 | 2.3 | 2.5 | 1.7 | 2.0 | / |
| Phenoxyethanol | / | / | / | 0.3 | 0.5 | 0.4 | / | / | 0.3 | 0.5 |
| Microdry | 33.0 | / | 36.9 | / | / | / | 20.0 | / | 33.3 | 30.0 |
| REACH ® 301 | / | 22.0 | / | 26.0 | 33.0 | 30.0 | / | 24.0 | / | / |
| Tocopheryl acetate | / | 0.5 | / | / | / | 0.5 | / | / | 0.3 | / |
| α-(2-Ethylhexyl)glycerol ether | / | 0.5 | / | 0.2 | / | 0.2 | / | / | 0.3 | / |
| Triethyl citrate | / | / | / | 0.4 | / | 0.2 | / | / | / | 0.3 |
| Cosmacol ® EMI | / | 2.9 | / | 2.7 | / | / | / | / | 2.0 | 3.0 |
| Cooling Agent | 0.5 | / | / | / | 0.5 | / | 0.5 | / | / | / |
| Isopulegol | / | / | 0.5 | / | 0.5 | / | / | / | 0.5 | / |
| Perfume | 1.0 | 1.0 | 1.0 | 1.0 | 1.0 | 1.0 | 1.0 | 1.0 | 1.0 | 1.0 |
| Water | ad 100 | ad 100 | ad 100 | ad 100 | ad 100 | ad 100 | ad 100 | ad 100 | ad 100 | ad 100 |

The water-in-oil emulsions presented in Examples 3-12 were mixed in a 20:80% ratio with n-butane as propellant gas and introduced into an aluminum can that was coated internally with an epoxy-phenol lacquer and was equipped with the Ariane M valve, obtainable from the Seaquist Perfect Company, and a valve plate coated internally with Microflex lacquer.

| | Example No. | | | | | | |
|---|---|---|---|---|---|---|---|
| | 13 | 14 | 15 | 16 | 17 | 18 | 19 |
| Dow Corning 345 Fluid | 0.82 | 0.36 | 0.72 | 0.82 | 0.82 | 0.82 | 0.82 |
| Dow Corning 200 Fluid, 5 cst | / | / | / | / | / | / | / |
| 1,2-Propylene glycol | 1.82 | 1.0 | 2.0 | 1.0 | 1.0 | 1.0 | 1.0 |
| Dipropylene glycol | / | / | / | / | / | / | / |
| Dow Corning 5225 C | 2.8 | 2.8 | 5.6 | 2.8 | 2.8 | 2.8 | 2.8 |
| Ethylhexyl palmitate | 0.46 | / | / | / | / | / | / |
| Phenoxyethanol | 0.1 | 0.1 | 0.2 | 0.1 | 0.1 | 0.1 | 0.1 |
| Microdry | 6.6 | / | 13.2 | 6.6 | 6.6 | 6.6 | 6.6 |
| REACH ® 301 | / | 6.6 | / | / | / | / | / |
| Tocopheryl acetate | / | / | / | / | / | / | / |
| α-(2-Ethylhexyl)glycerol ether | / | / | / | / | / | / | / |
| Triethyl citrate | / | / | / | / | / | / | / |
| Finsolv ® TN | / | 0.92 | 1.84 | 0.46 | 0.46 | 0.46 | 0.46 |
| Cooling Agent | 0.5 | / | / | / | / | / | / |
| Dow Corning 1501 Fluid | / | / | / | / | 1.0 | / | 1.0 |
| Perfume | 1.0 | 1.0 | 2.0 | 1.0 | 1.0 | 1.0 | 1.0 |
| Panthenol | / | / | / | / | / | 0.3 | / |
| N,N′-Bis(2-hydroxyethyl)urea | / | / | / | / | / | / | 0.5 |
| Water | 7.4 | 8.22 | 16.44 | 8.22 | 8.22 | 8.22 | 8.22 |
| n-Butane | 78.5 | 79 | 58 | 79 | 78 | 78.7 | 77.5 |

The compositions presented in Examples 13-19 were introduced into an aluminum can that was coated internally with an epoxy-phenol lacquer and was equipped with the Ariane M valve, obtainable from the Seaquist Perfect Company, and a valve plate coated internally with Microflex lacquer.

List of raw materials used.

| | | |
|---|---|---|
| Dow Corning 345 | Cyclomethicone | Dow |
| Fluid | (decamethylcyclopentasiloxane, | Corning |
| | dodecamethylcyclohexasiloxan) | |
| Dow Corning 200 | Dimethicone | Dow |
| Fluid, 5 cst | | Corning |
| Dow Corning 5225 C | Cyclomethicone, PEG/PPG-18/18 | Dow |
| Formulation Aid | Dimethicone at a 9:1 weight ratio | Corning |
| Microdry ® | Aluminum chlorohydrate | Reheis |
| Dow Corning 1501 | Cyclomethicone, dimethiconol | Dow |
| Fluid | | Corning |
| REACH ® 301 | Aluminum sesquichlorohydrate | Reheis |
| Cosmacol ® EMI | Di-C₁₂₋₁₃ alkyl malate | Nordmann |
| | | Rassmann |
| Cooling Agent | Menthyl lactate, menthyl | — |
| | glycolate, menthol (1:1:0.4) | |

1. A perspiration-inhibiting and/or deodorizing cosmetic product, comprising
a water-in-oil emulsion comprising at least one antiperspirant and/or deodorant active substance,
at least one propellant, and
an aerosol dispensing apparatus comprising a valve,
wherein the parts of the valve of the dispensing apparatus that come into contact with the emulsion are made of nonmetallic materials.

2. The cosmetic product according to claim 1, wherein the valve comprises a valve cone and/or a flexible element having return characteristics, which is/are coated with a lacquer or with a polymeric plastic A.

3. The cosmetic product according to claim 2, wherein the flexible element having return characteristics is embodied as a spiral spring or helical compression spring.

4. The cosmetic product according to claim 2, wherein the flexible element having return characteristics is embodied integrally with the valve cone and comprises flexible legs.

5. The cosmetic product according to claim 1, wherein the valve comprises a flexible element having return characteristics and/or a valve cone made of at least one plastic B.

6. The cosmetic product according to claim 5, wherein the plastic B is an elastomeric plastic.

7. The cosmetic product according to claim 1, wherein the dispensing apparatus comprises a springless valve.

8. The cosmetic product according to claim 1, wherein the valve comprises an internally lacquered valve plate, the lacquer coating and valve material being mutually compatible.

9. A perspiration-inhibiting and/or deodorizing cosmetic product, comprising
a water-in-oil emulsion comprising at least one antiperspirant and/or deodorant active substance and an oil phase of 1-60 wt %, based on the total weight of the emulsion,
at least one propellant, and
an aerosol dispensing apparatus comprising a valve,
wherein the parts of the valve and the dispensing apparatus that come into contact with the emulsion are made of nonmetallic materials.

10. The cosmetic product according to claim 9, wherein at least 90 wt % of the oil phase is made up of oil components that are liquid at 20° C.

11. The cosmetic product according to claim 9, wherein the oil phase is made of components that are selected from the group consisting of decamethylcyclopentasiloxane, dodecamethylcyclohexasiloxane, hexamethyldisiloxane (L₂), octamethyltrisiloxane (L₃), decamethyltetrasiloxane (L₄), two- and three-member mixtures of L₂, L₃ and/or L₄, 2-ethylhexyl palmitate, hexyldecyl laurate, 2-ethylhexyl stearate, 2-ethylhexyl laurate, isopropyl myristate, isopropyl palmitate, C₁₂-C₁₅ alkyl benzoate, C₁₂-C₁₅ alkyl lactate, di-C₁₂-C₁₃ alkyl malate, PPG-14 butyl ether, isododecane, isohexadecane, isoeicosane, polyisobutene, polydecenes, and mixtures thereof.

12. The cosmetic product according to claim 9, wherein the oil phase comprises an oil mixture that contains more than 50 wt % of at least one volatile cyclic or linear silicone oil.

13. The cosmetic product according to claim 9, in which the oil phase is made up of oil components in which 5-50 wt %, of the components exhibit a refractive index n_{D} from 1.43-1.51 at 20° C. (measured at λ=589 nm).

14. The cosmetic product according to claim 9, wherein at least one water-in-oil emulsifion comprises an emulsifier, selected from the group consisting of PEG-x Dimethicone (where x=2-20), Bis-PEG-y Dimethicone (where y=3-25), PEG/PPG a/b Dimethicone (where a and b, mutually independently, denote numbers from 2-30), Bis-PEG/PPG-c/d Dimethicone (where c and d, mutually independently, denote numbers from 10-25), and Bis-PEG/PPG-e/f PEG/PPG g/h Dimethicone (where e, f, g, and h, mutually independently, denote numbers from 10-20.

15. The cosmetic product according to claim 1, wherein the water-in-oil emulsion contains a water phase of 40-99 wt %, based on the total weight of the emulsion.

16. The cosmetic product according to claim 1, wherein the at least one antiperspirant active substance is present in a quantity of 15-55 wt %, based on the total weight of the water-in-oil emulsion.

17. The cosmetic product according to claim 1, wherein at least one deodorant active substance is present in a quantity of 0.1-10 wt % based on the total weight of the water-in-oil emulsion.

18. The cosmetic product according to claim 14, wherein at least one water-in-oil emulsifier is present in a quantity of 0.5-5 wt %, based on the total weight of the emulsion.

19. The cosmetic product according to claim 1, wherein at least one water-soluble polyvalent C₂-C₉ alkanol having 2-6 hydroxyl groups and/or water-soluble polyethylene glycol having 3-20 ethylene oxide units is present in a quantity of 0.5-25 wt %, based on the entire water-in-oil emulsion.

20. The cosmetic product according to claim 1, said product comprising 10-90 wt % propellant, based on the total weight of the water-in-oil emulsion and the propellant.

21. The cosmetic product according to claim 10, wherein 5-50 wt % of the oil components that are liquid at 20° C. are selected from the group consisting of isopropyl myristate, isopropyl palmitate, isohexadecane, isoeicosane, PPG-14 butyl ether, PPG-15 butyl ether, 2-hexyldecanol, isostearyl benzoate, dimethicone PEG/PPG-20/23 benzoate, PPG-53 butyl ether, isostearyl lactate, isostearyl palmitate, hexyldecyl laurate, mixtures of hexyldecanol and hexyldecyl laurate, isocetyl palmitate, 2-octyldodecanol, polydecenes, isocetyl stearate, 2-ethylhexyl stearate, hexyldecyl stearate, 16-methyl-1-heptadecanol, diethylhexylcyclohexane, 2-ethylhexyl laurate, benzyl laurate, C₁₂-C₁₅ alkyl benzoate, octyldodecyl benzoate, C₁₂-C₁₅ alkyl lactate, dimethicone PEG-8 benzoate, PPG-5-buteth-7, PPG-2-isodeceth-12, polyphenylmethylsiloxanes, phenyltrimethicone, PPG-2-ceteareth-9, isostearyl isostearate, di-C₁₂-C₁₃ alkyl malate, isododecane, polyisobutene, and glycereth-7 benzoate and mixtures thereof.

22. A perspiration-inhibiting and/or deodorizing cosmetic product comprising
a water-in-oil emulsion comprising at least one antiperspirant and/or deodorant active substance and at least one dimethiconol,
at least one propellant, and
an aerosol dispensing apparatus comprising a valve,
wherein the parts of the valve of the dispensing apparatus that come into contact with the emulsion are made of nonmetallic materials.

23. The cosmetic product according to claim 1, wherein the water-in-oil emulsion comprises at least one moisture-donating active substance selected from the group consisting of panthenol, pantolactone, deoxy sugars, rhamnose, fucose, polysaccharides that contain at least one deoxy sugar module, urea, N,N′-bis(2-hydroxyethyl)urea, betaine (Me₃N⁺—CH₂—COO⁻), glycosaminoglycans, hyaluronic acid, dextran, dextran sulfate, chondroitin 4-sulfate, and chondroitin 6-sulfate, as well as mixtures thereof.
